(19) United States
(12) Patent Application Publication
(10) Pub. No.: US 2020/0309711 A1
(43) Pub. Date: Oct. 1, 2020

(54) MATERIAL TESTING DEVICE AND SYSTEM

(71) Applicant: UNIVERSITY OF ALASKA ANCHORAGE, Anchorage, AK (US)

(72) Inventors: ZHAOHUI YANG, Anchorage, AL (US); LIN LI, Anchorage, AK (US)

(21) Appl. No.: 16/828,424

(22) Filed: Mar. 24, 2020

Related U.S. Application Data
(60) Provisional application No. 62/823,274, filed on Mar. 25, 2019.

(51) Int. Cl. G01N 21/88; G01N 25/72; G01N 13/02; G01N 33/24; G01B 11/16; H04N 5/33

(52) U.S. Cl. CPC G01N 21/88; G01N 25/72; H04N 5/33; G01N 33/24; G01B 11/16; G01N 13/02

(57) ABSTRACT

A material testing system can be used for measuring material properties. The system can include an inner cell and an outer cell that cooperate to at least partially define a first internal volume. The inner cell can at least partially define a second volume therein and is configured to receive a material sample. A first plurality of cameras that are configured to detect visible light surround the circumference of the inner cell. A second plurality of cameras that are configured to detect infrared light surround the circumference of the inner cell. A thermocouple can provide a reference temperature to calibrate thermal data from the second plurality of cameras. The first plurality of cameras, the second plurality of cameras, and the thermocouple can provide data to a computing device. A pattern on the material sample can enable the computing device to track movement of discrete points on the material.

### CROSS-REFERENCE TO RELATED APPLICATION

This application claims priority to and the benefit of U.S. Provisional Application No. 62/823,274, filed Mar. 25, 2019, the entirety of which is hereby incorporated by reference herein.

### FIELD

This application relates generally to systems for testing material properties and, more specifically, to systems for using a plurality of cameras to detect strain in the materials.

### BACKGROUND

Engineering materials such as soils, rocks, and concrete are essential for engineering construction of infrastructures such as roads, dams, runways, pipelines, building foundations, etc. Frost susceptible soils (e.g., soils containing silt and clays) extensively exist in Alaska and other cold climate territories. When underlain by frost susceptible soils, infrastructures are vulnerable to ground heave during freezing in winter and excessive settlement and weakening during thawing in spring. One-dimensional frost heave tests are used to characterize some aspects of the material engineering behavior. However, the conventional frost heave test apparatus (ASTM 2013) is only capable of measuring the total heave and thaw settlement amount at a certain temperature gradient. It is incapable of capturing the temperature distribution along the specimen, and it is unable to record the development of ice lenses in the specimen during testing. Accordingly, a more comprehensive test is desirable.

### SUMMARY

Disclosed herein is a material testing device and system. The material testing device, which, in some embodiments can be capable of simultaneous monitoring of volumetric change, local strain development, and pore pressure variation, can help characterize (in detail) the heave of frost susceptible soils during freezing and settlement of the same during thawing. In this material testing device, the soil pore-water pressure (u_{w}) variation during one- or three-dimensional freezing can be measured using self-developed high-capacity tensiometers, and the soil deformation (i.e. frost heave or thaw settlement) during freezing is monitored by a multi-camera photogrammetric method. With this material testing device, the behavior of different frost susceptible soils during freezing or thawing can be evaluated through one- or three-dimensional frost heave/thaw consolidation tests under different water access and temperature gradient conditions. The information gathered by the material testing device can provide insight into the fundamental driving forces in frost heave and shed lights on solutions of frost-related engineering issues. A soil and other material testing device and system with pore-water pressure measuring and full-field deformation monitoring capabilities, disclosed herein, can be used for research on understanding the fundamental mechanisms of soils and assess the effectiveness of engineering measures for application in geotechnical and transportation fields. These new capabilities are not only applicable for frozen soils, but also for unfrozen soils and geological or other materials, and have broad applications in the geotechnical engineering.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIG. 1 is a schematic of a material testing device and system in accordance with the present invention;

FIG. 2 is a schematic of the material testing device and system as in FIG. 1 illustrating various further aspects;

FIG. 3 is a schematic of a top view of the material testing device and system as in FIG. 1;

FIG. 4 is a schematic of the material testing device and system as in FIG. 1 illustrating yet further aspects;

FIG. 5A illustrates first material sample surface pattern for use with the material testing device and system as in FIG. 1;

FIG. 5B illustrates second material sample surface pattern for use with the material testing device and system as in FIG. 1;

FIG. 6A illustrates a 3D dot matrix generated from the material surface pattern as in FIG. 5A;

FIG. 6B illustrates a circumferential mesh surface generated from the dot matrix as in FIG. 6A;

FIG. 6C illustrates an enclosed mesh volume generated including the circumferential mesh surface as in FIG. 6B;

FIG. 6D illustrates some tetrahedral components of a tetrahedral volumetric mesh generated from the enclosed mesh volume as in FIG. 6C.

FIG. 7A illustrates a surface mesh as in FIG. 6B of a material as it changes over time;

FIG. 7B illustrates a heat map of strain generated from the surface mesh as in FIG. 7A.

FIG. 7C is an image of the material sample from which the surface mesh of FIG. 7A was generated;

FIG. 8 illustrates a system comprising a computing device for use with the material testing device and system as in FIG. 1; and

FIG. 9 illustrates a system comprising an exemplary computing device for use with the material testing device and system as in FIG. 1.

### DETAILED DESCRIPTION

The present invention can be understood more readily by reference to the following detailed description and appendix, which include examples, drawings, and claims. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an,” and “the” include plural referents unless the context clearly dictates otherwise. Thus, for example, reference to “a thermocouple” or to “a camera” can include two or more such thermocouples or cameras unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used herein, the terms “optional” or “optionally” mean that the subsequently described event or circumstance may or may not occur, and that the description includes instances where said event or circumstance occurs and instances where it does not.

### INTRODUCTION TO THE TECHNOLOGY

Triaxial tests have been used to characterize both saturated and unsaturated soils. For saturated soils, the conventional triaxial test equipment, with the volume change measurement capability and with/without soil pore-water pressure measurement capability, can be used for soil behavior investigation. However, the conventional triaxial test equipment cannot be directly used for unsaturated soil behavior investigation due to the difficulties in the measurement of volume change/deformation and pore-water pressure of unsaturated soils. In 1961, Bishop and Donald developed the suction-controlled triaxial test apparatus in which the soil pore-water pressure was controlled using the axis-translation technique, and the soil volume change was measured using a double cell technique. However, this type of equipment is sophisticated and therefore expensive, and the triaxial test is very time-consuming due to the low permeability of unsaturated soils. In 2015, Li and Zhang developed a triaxial test apparatus for unsaturated soil behavior characterization in which the soil pore-water pressure during testing was measured using high-capacity tensiometers and soil volume change/deformation was measured using a photogrammetry-based method. However, because this system uses a single camera for image capturing, and due to a certain time period being required for image capturing, the soil deformation cannot be continuously measured. Besides this, the triaxial test apparatus is not suitable for soil frost heave behavior characterization due to lack of a temperature control device.

The fundamental mechanism of frost heave and thaw weakening is moisture migration in the fine-grain soils driven by the differences in negative pore-water pressure (this phenomenon is typically known as capillary rise) generated during freezing. Significant research effort has been dedicated to understanding various factors affecting the frost heave behavior of soils such as soil mineral type, grain-size distribution, overburden pressure, quantified soil heave rate, and soil frost susceptibility. In order to facilitate the required frost heave test, different types of frost heave cells have been developed, in which the soil weights before and after the frost heave process are manually measured to determine the water intake. Alternatively, the water intake during freezing can be monitored using a differential pressure transducer. However, in these frost heave cells, the soil negative pore-water pressure, during freezing that drives water migration, is not directly used in assessing soil frost susceptibility due to difficulties in direct pore-water measurement. In other words, the mechanism of the freezing process associated ice lens formation process is still not fully understood due to the lack of the specific details of water transformation. Besides this, in the conventional frost heave test, only the total heave amount is monitored by displacement transducers (e.g. LVDT or laser). Using such techniques, due to ice lens formation and a lack of a method for a full-field deformation monitoring, the resultant non-uniform soil deformation cannot be accurately examined. Consequently, such techniques leave many uncertainties in frost susceptibility assessment. Accordingly, the material testing device and system as disclosed herein can overcome one or more of the above-disclosed limitations.

### The Disclosed Material Testing Devices and Systems

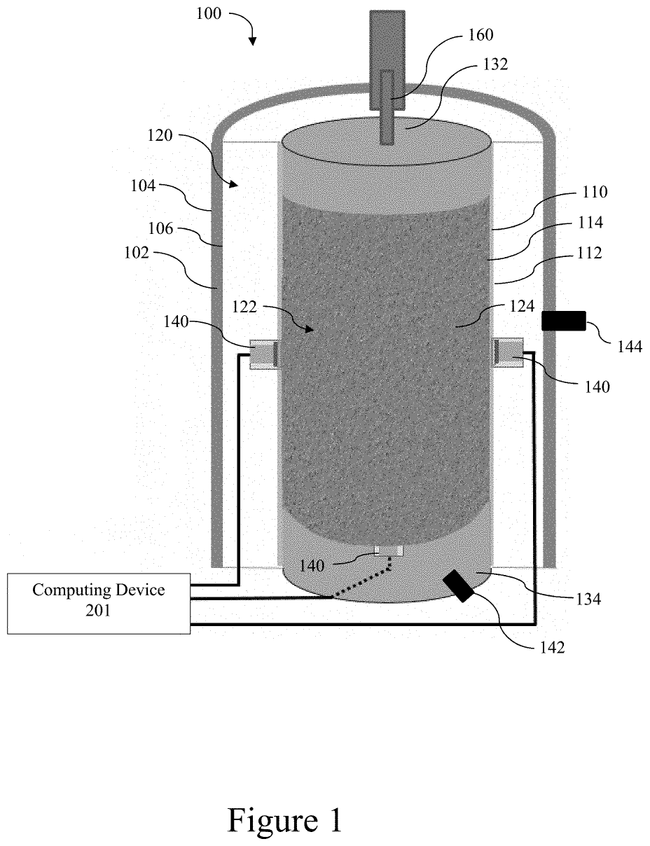

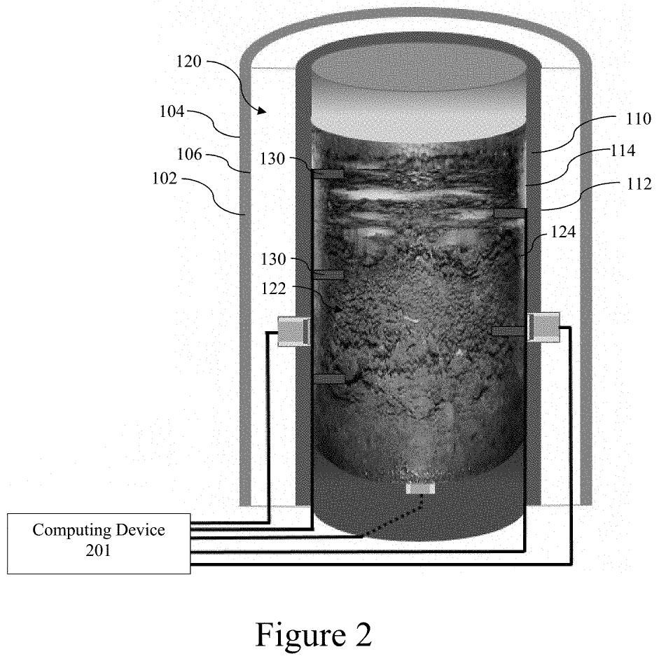

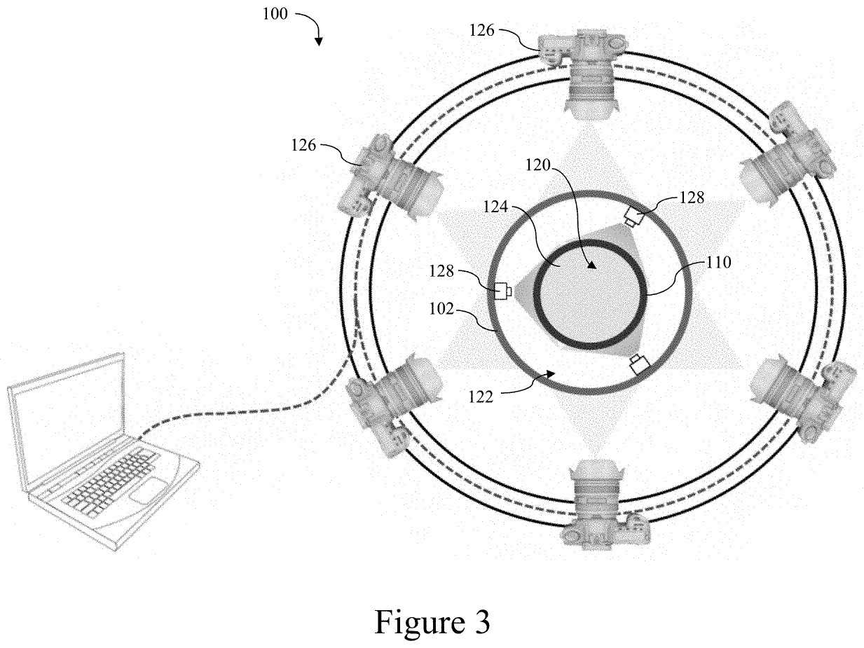

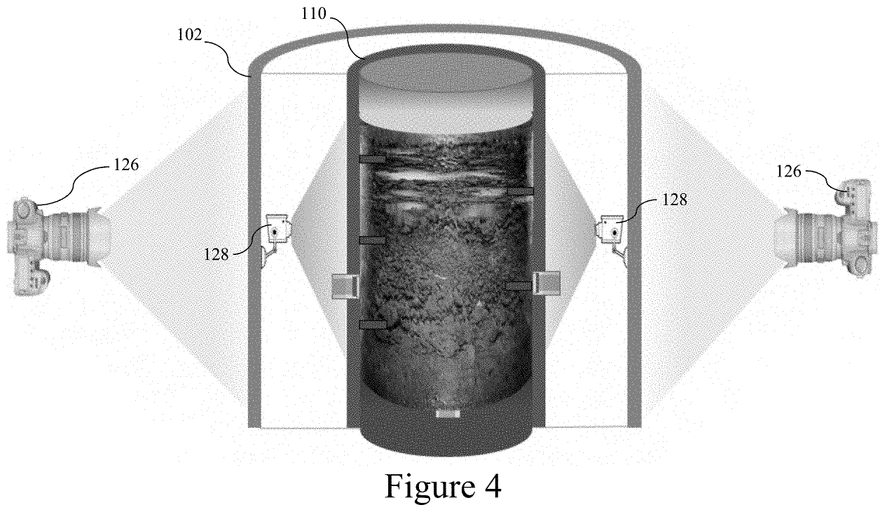

Referring to FIGS. 1-4, a material testing device 100 can comprise a transparent outer cell 102 having an outer surface 104 and an inner surface 106. The outer cell can optionally comprise acrylic material. The material testing device 100 can further comprise a transparent inner cell 110 having an outer surface 112 and an inner surface 114. The inner surface 106 of the outer cell 102 and outer surface 112 of the inner cell 110 can cooperate to at least partially define a first interior volume 120. The first interior volume 120 can be configured to receive a pressure controlled air or transparent fluid.

The inner surface 114 of the inner cell 110 can at least partially define a second interior volume 122. The second interior volume can be configured to receive a material sample 124. The transparent inner cell 110 can engage an outer surface of the material sample. The transparent inner cell 110 can comprise a flexible material, such as, for example, a latex membrane. In this way, the cell 110 can allow for expansion and contraction of the material sample 124. In further embodiments, the transparent inner cell can comprise a rigid, non-flexible material so that the material can expand and contract only in one dimension, for example, in order to test frost heave. The transparent inner cell can be impermeable to water.

A first plurality of cameras 126 can be disposed about a perimeter of the transparent inner cell 110. Optionally, the first plurality of cameras 126 can be disposed outside of the transparent outer cell 102. According to various aspects, six to eight cameras can be evenly spaced about the perimeter in order to capture the entire circumference of the material sample. It is contemplated that the first plurality of cameras 126 can be configured to detect light in the visible spectrum. Cameras 126 can be commercially available single lens reflex cameras, such as, for example, Nikon D7000 cameras.

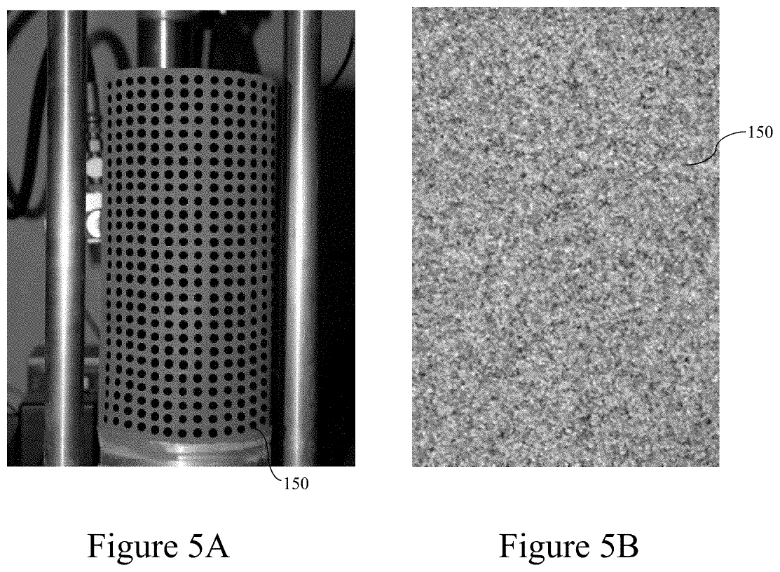

The material sample 124 can have a pattern 150 on its circumferential surface so that the first plurality of cameras 126 can capture changes in the pattern that correspond to spatial changes between respective points on the material sample's surface. In some embodiments, the material surface can have an inherent pattern, such as, for example, a sample comprising a sand texture, as shown in FIG. 5B. In further embodiments, the pattern 150 can be applied to the material sample's surface, as shown in FIG. 5A. For example, a dot matrix can be printed on the material sample's surface. The dot matrix can be a square grid, triangular grid, hexagonal grid, or any other suitable pattern. The dot matrix can optionally be screen printed. According to one aspect, a negative pattern can be created, such as, for example, a flexible sheet comprising a grid of holes. The pattern can be wrapped around the circumference of the material sample, and paint can be sprayed onto the pattern-wrapped sample. When the pattern is removed, dots of paint can remain where the pattern's holes were positioned. The dot matrix can use various patterns, but dots oriented as a pattern of squares or triangles can simplify subsequent calculations.

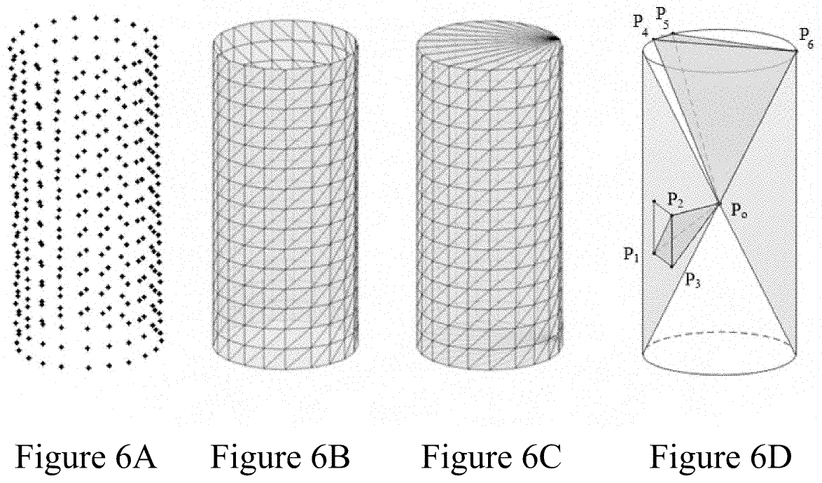

Referring also to FIGS. 6A-6D, the dot matrix or other surface pattern 150 can be used to construct a 3D point cloud that approximates the material sample's shape at a given time. Multiple point clouds can be created at select intervals in order to track changes in the material sample's shape over time. Using 3D coordinates of the dots on the material sample's surface (FIG. 6A), a triangular mesh can be constructed, as shown in FIG. 6B. The material sample can then be defined according to a tetrahedral mesh so that volumetric properties can be determined. For example, for a cylindrical material sample, a top surface and a bottom surface can be defined as meshes (e.g., as shown in FIG. 6C, the top surface mesh is defined by uppermost points of the dot matrix and comprising a plurality of triangles each defined by a first point as a common vertex, for example, P₆, and a pair of respective circumferentially adjacent points, for example, P₄ and P₅, of the remaining points). Accordingly, the exterior of the material sample can be approximated as a volume enclosed by the three-dimensional mesh comprising the circumferential surface mesh, the top disc surface mesh, and the bottom surface mesh. A point in the interior of the material sample can be selected (e.g., the centroid, P₀), and a plurality of tetrahedrons can be defined by three points of the surface matrix (e.g., P₁, P₂, and P₃) on the circumferential surface and the center point, as shown in FIG. 6D. Similarly, the points from the top and bottom mesh can define three points (e.g., P₄, P₅, and P₆) of respective tetrahedrons, with the fourth point being the interior point of the material sample. Therefore, a total volume of the space within the enclosed mesh can be the sum of all of the volumes of the tetrahedrons, which can approximate the total volume of the material sample. In this way, volumetric calculations can be determined from images of the dot matrix on the material sample. MATLAB software packages or various other computational software packages may be used to create and use the mesh as disclosed herein.

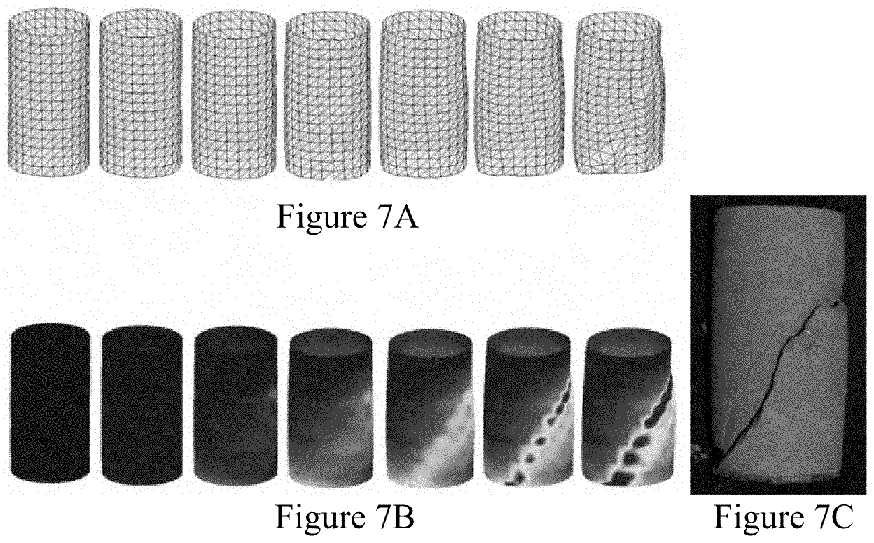

FIGS. 7A-7C illustrates an application of the surface mesh to a material sample in a test of strain over time. FIG. 7A illustrates changes in the surface mesh over time, corresponding with changes in the surface of the material sample. FIG. 7B illustrates a heat map of axial strain in the material sample's surface over time. FIG. 7C illustrates an image of the material sample at the end of the test duration.

Referring again to FIGS. 1-4, a second plurality of cameras 128 can be disposed about the circumference of the inner cell. Optionally, the second plurality of cameras 128 can be disposed within the outer cell 102. According to some embodiments, three to five cameras 128 can be evenly spaced around the circumference of the material sample. The second plurality of cameras 128 can be configured to detect light in the infrared spectrum. That is, the second plurality of cameras 128 can determine the temperature of the material sample 124 at various locations across its surface. In exemplary aspects, it is contemplated that conventional software packages (e.g., MatLab, C++, and the like) can be used for the infrared image processing as disclosed herein.

In some embodiments, the first plurality of cameras 126 can be spaced so that each camera's field of capture overlaps with those of respective adjacent cameras 126. In this way, an entirety of the sample's circumferential surface can be captured by the first plurality of cameras 126. Moreover, the overlapping portions of cameras can be used to orient a first camera's image capture with respect to an adjacent camera's image capture. Unlike the plurality of cameras 126, no field of capture overlap is required for adjacent cameras 128.

The first plurality of cameras 126 can be spaced from the sample so that their combined field of view captures the material sample's circumferential surface along its entire axial length. In further embodiments, it is contemplated that the first plurality of cameras 126 can include multiple rows of vertically (or axially) spaced cameras in order to capture the entirety of the material sample's circumferential surface along its axial length. The second plurality of cameras 128 can be similarly configured to capture the entirety of the material sample's circumferential surface along its axial length.

In further embodiments, a single plurality of cameras 126 can be used to detect both visible light and infrared images. In such embodiments, the single plurality of cameras can comprise image sensors that are capable of capturing both visible-light and infrared images, and the second plurality of cameras 128 can be excluded.

One or more thermocouples 130 can be in contact with a surface of the material sample in order to accurately determine the temperature of the material sample's surface at each thermocouple's respective location. In this way, the thermocouples 130 can act as a reference to calibrate the thermal data received from the plurality of infrared cameras 128. That is, a computing device, as further disclosed herein, can receive temperature data from the thermocouples 130 and use the thermocouple temperature data to define the accurately measured reference temperature at the measured locations, and temperature data from the infrared cameras at locations spaced from the thermocouples can be measured with respect to that reference.

Accordingly, a temperature map can be created according to the following process. The distance between each infrared camera 128 and the specimen can be determined, as well as the central angle defining the scope of each image coverage. An idealized coordinate system for all points on the specimen can then be constructed for each infrared image from each camera 128. Three dimensional coordinates of all points on the specimen surface can then be determined. In a further embodiment, the thermal images can be stitched together, and the stitched images can then be mapped to the spatial 3D model in order to provide a coordinate system for the thermal image data. Conventional image correlation techniques can be used to stitch the thermal images together.

The temperatures at each point can then be corrected based on the thermocouples 130. For example, if the temperature measurement from the camera 128 is four degrees lower than the thermocouple 130, the temperature measurement for the point and the surrounding points can be adjusted by four degrees. For embodiments including two or more thermocouples, the system can interpolate an adjustment factor between the thermocouples. Finally, the three dimensional coordinates of all points in the idealized coordinate system can then be transformed into the global coordinates system that incorporates the material specimen deformation, as herein described with reference to FIGS. 6A-6D. In this way, a full-field material temperature, in conjunction with deformation, can be used to characterize material behavior. In use, it is contemplated that the thermocouples can translate or otherwise move with the sample under load. Thus, the three-dimensional position and corresponding temperature of each thermocouple can be updated for each measurement.

Referring to FIGS. 1-4, a top plate 132 can engage a top surface of the material sample, and a bottom plate 134 can engage a bottom surface of the material sample. The top plate 132 and bottom plate 134 can cooperate with the inner surface 112 of the inner cell 110 to define the second interior volume 122. The top plate 132 and bottom plate 134 can apply a temperature gradient to the material sample 124. For example, the top plate 132 can apply a subfreezing temperature to the top of the material sample 124, and the bottom plate can apply a non-freezing temperature to the bottom of the material sample 124. For example, heaters embedded in the top and bottom plates can apply a known heat flux to the material sample. In further embodiments, the top and bottom plates can further comprise one or more embedded thermocouples, and a controller in communication with the thermocouple(s) can maintain the top and bottom plates at desired temperatures. In yet further embodiments, coolant paths and/or thermoelectric heating and cooling devices can be incorporated into the top and bottom plates in order to provide desired heating and cooling conditions. Coolant for said coolant paths can be supplied at a desired temperature from a temperature-regulated reservoir.

At least one tensiometer 140 can operably couple to the inner surface 114 of the inner cell 110 (therefore, in contact with the outer surface of the material sample) so that it can measure the pore-water pressure of the material sample. One or more tensiometers 140 can further operably couple to the material sample 124, for example, at the material sample's bottom surface. The one or more tensiometers 140 can be high-capacity tensiometers (HCTs) as are known in the art. In some embodiments, the tensiometer can measure negative pore water pressure (i.e., suction) up to 15 bar (1.5 MPa).

In use, it is contemplated that the devices and systems disclosed herein can be used to test frost heave and facilitate full-field soil temperature variation and direct soil pore-water pressure measurement. The top plate can apply a sub-freezing temperature, and the bottom plate can apply a nonfreezing temperature, or vice versa. The pore-water pressure variation can be measured using the tensiometers. The plurality of cameras 126 can capture deformation of the sample. For example, the plurality of cameras 126 can detect axial deformation of segments along the axis of the material sample (e.g., the change in distance between two adjacent points along the axis relative to the original distance between the two points). Accordingly, the disclosed material testing device and system can be used to detect localized strain and, thus, characterize non-uniform axial deformation, as can be common for frost heave. Further, the image data from the cameras can be used to determine change in relative position (and, thus strain) between any two points on the material sample, and, therefore, can measure three-dimensional strain.

Further, it is contemplated that the material testing device 100 can simulate various water conditions. For example, in nature, situations can occur in which the soil is not subject to ground water supply from the water table. Accordingly, the material testing device 100, as disclosed herein, can simulate said condition by not providing water to the material sample. Conversely, soil in nature can be subject to receiving water from the ground water table. Accordingly, for frost heave testing, one water condition can be open-water access where water intake at the bottom of a soil sample is allowed to infiltrate the bottom of the material sample. For example, the bottom plate 134 have an inlet 142 for receiving water intake. The bottom plate can further comprise a porous stone that allows water to disperse evenly throughout the bottom plate. The water intake can be provided at a controlled temperature, such as, for example, near the freezing point of the water. In alternative configurations, the water inlet 142 can be provided at the top plate 132, for example. Such an alternative configuration can be used when the bottom plate 134 is cold and the top plate 132 is hot.

According to one aspect, the material testing device 100 can be used for measuring triaxial mechanical behavior of the material sample. The second cell can comprise a flexible material. The top and bottom plates 132, 134 can apply an axial load to the material sample 124, for example, via one or more hydraulic pistons 160. An inlet 144 to the first volume can receive air or another transparent fluid therethrough. Accordingly, the first interior volume 120 can be pressurized in order to apply a confining stress to the inner cell 110 and, thus, the material sample. Such a confining stress can simulate a situation in which the soil specimen is in the ground and receives lateral loads from adjacent soil. According to some aspects, confining pressures can be from 0 to 1200 kPa.

According to one aspect, the material sample 124 can have a cylindrical or generally cylindrical profile. The cylindrical material sample can have a central axis that is longer than its diameter. Although this disclosure refers to a sample's circumference, it should be understood that samples are not limited to a cylindrical profile. The circumference should be interpreted to include a sample's exterior surface which can be captured by a camera that is revolved 360 degrees about an axis through the material sample and along the length of said axis. According to some aspects, the material sample can be a soil specimen.

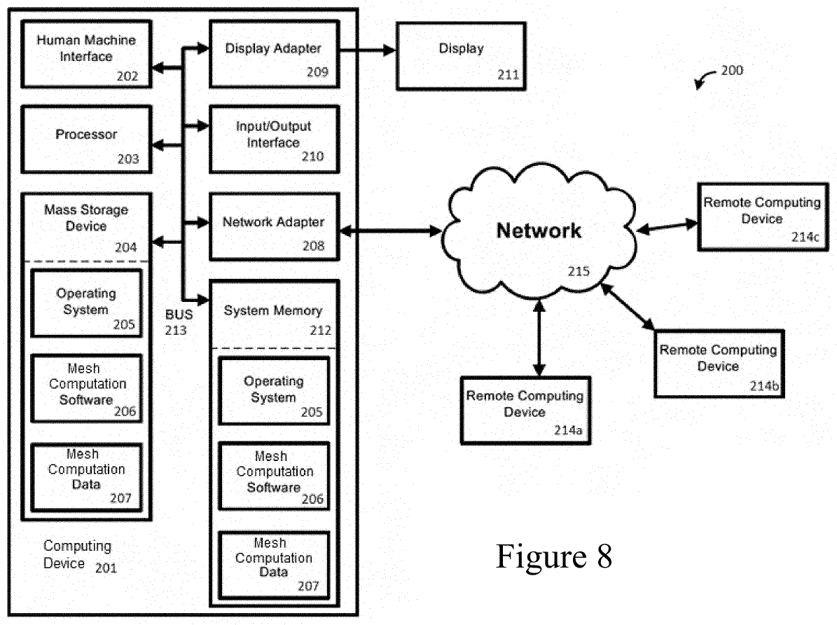

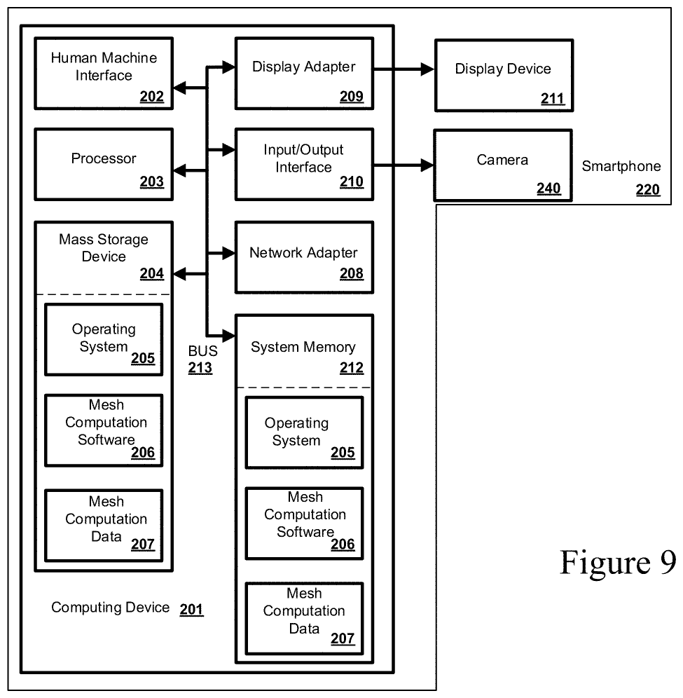

FIG. 8 shows an exemplary computing system 200 that can be used with the material testing device 100. Computing system 200 can include a computing device 201 and a display 211 in electronic communication with the computing device. In some optional embodiments, and as shown in FIG. 9, a smart phone 220 (or other remote computing device, such as a tablet) can comprise both the computing device 201 and the display 211. Alternatively, it is contemplated that the display 211 can be provided as a separate component from the computing device 201. For example, it is contemplated that the display 211 can be in wireless communication with the computing device 201, thereby allowing usage of the display 211 in a manner consistent with that of the display of the smartphone as disclosed herein.

The computing device 201 may comprise one or more processors 203, a system memory 212, and a bus 213 that couples various components of the computing device 201 including the one or more processors 203 to the system memory 212. In the case of multiple processors 203, the computing device 201 may utilize parallel computing.

The bus 213 may comprise one or more of several possible types of bus structures, such as a memory bus, memory controller, a peripheral bus, an accelerated graphics port, and a processor or local bus using any of a variety of bus architectures.

The computing device 201 may operate on and/or comprise a variety of computer readable media (e.g., non-transitory). Computer readable media may be any available media that is accessible by the computing device 201 and comprises, non-transitory, volatile and/or non-volatile media, removable and non-removable media. The system memory 212 has computer readable media in the form of volatile memory, such as random access memory (RAM), and/or non-volatile memory, such as read only memory (ROM). The system memory 212 may store data such as mesh computation data 207 and/or program modules such as operating system 205 and mesh computation software 206 that are accessible to and/or are operated on by the one or more processors 203.

The computing device 201 may also comprise other removable/non-removable, volatile/non-volatile computer storage media. A mass storage device 204 may provide non-volatile storage of computer code, computer readable instructions, data structures, program modules, and other data for the computing device 201. The mass storage device 204 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 204. An operating system 205 and the mesh computation software 206 may be stored on the mass storage device 204. One or more of the operating system 205 and the mesh computation software 206 (or some combination thereof) may comprise program modules and the mesh computation software 206. Mesh computation data 207 may also be stored on the mass storage device 204. The mesh computation data 207 may be stored in any of one or more databases known in the art. The databases may be centralized or distributed across multiple locations within the network 215.

A user may enter commands and information into the computing device 201 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 203 via a human machine interface 202 that is coupled to the bus 213, but may be connected by other interface and bus structures, such as a parallel port, game port, an IEEE 1394 Port (also known as a Firewire port), a serial port, network adapter 208, and/or a universal serial bus (USB).

A display 211 may also be connected to the bus 213 via an interface, such as a display adapter 209. It is contemplated that the computing device 201 may have more than one display adapter 209 and the computing device 201 may have more than one display 211. A display 211 may be a monitor, an LCD (Liquid Crystal Display), light emitting diode (LED) display, television, smart lens, smart glass, and/or a projector. In addition to the display 211, other output peripheral devices may comprise components such as speakers (not shown) and a printer (not shown) which may be connected to the computing device 201 via Input/Output Interface 210. Any step and/or result of the methods may be output (or caused to be output) in any form to an output device. Such output may be any form of visual representation, including, but not limited to, textual, graphical, animation, audio, tactile, and the like. The display 211 and computing device 201 may be part of one device, or separate devices.

The computing device 201 may operate in a networked environment using logical connections to one or more remote computing devices 214a,b,c. A remote computing device 214a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 201 and a remote computing device 214a,b,c may be made via a network 215, such as a local area network (LAN) and/or a general wide area network (WAN). Such network connections may be through a network adapter 208. A network adapter 208 may be implemented in both wired and wireless environments. Such networking environments are conventional and commonplace in dwellings, offices, enterprise-wide computer networks, intranets, and the Internet. In further exemplary aspects, it is contemplated that the computing device 201 can be in communication with the remote computing devices 214a,b,c through a Cloud-based network.

Application programs and other executable program components such as the operating system 205 are shown herein as discrete blocks, although it is recognized that such programs and components may reside at various times in different storage components of the computing device 201, and are executed by the one or more processors 203 of the computing device 201. An implementation of the mesh computation software 206 may be stored on or sent across some form of computer readable media. Any of the disclosed methods may be performed by processor-executable instructions embodied on computer readable media.

The computing device 201 can receive data from the plurality of cameras 126, 128, thermocouples 130, tensiometers 140, and other collected data via wired or wireless communication. The computing device 201 can be configured to control heat input, regulate temperature at various locations, regulate pressure in the first interior volume 120, and deliver a controlled quantity of water to the material sample. The computing device 201 can further be configured to control various other aspects of experimentation, collect other relevant data, and perform various computations based on the collected data (e.g., generate meshes and calculate volume changes, as discussed herein).

The computing device can further be configured to calculate error according to the following equation:

Eₒᵥₑᵣₐₗₗ=f(c,n,r_{c},r_{f},dₒ,dᵢ,shₒ,shᵢ,s)

where,
Eₒᵥₑᵣₐₗₗ=overall error of the photogrammetric measurement method,
c=influence factor introduced by the camera used, dependent on camera and lens models and the associated calibration technique,
n=influence factor introduced by the number of images used for each deformation measurement,
r_{c}=influence factor introduced by an inaccurate estimation of the cell refractive index,
r_{f}=influence factor introduced by an inaccurate estimation of the fluid refractive index,
dₒ=influence factor introduced by an inaccurate estimation of the outer cell thickness,
dᵢ=influence factor introduced by an inaccurate estimation of the inner cell thickness,
shₒ=influence factor introduced by an inaccurate estimation of the outer cell shape,
shᵢ=influence factor introduced by an inaccurate estimation of the inner cell shape, and
s=influence factor introduced by geometric configuration of the testing system, dependent on size of the sample and radius and thickness of the inner and outer cells.

In addition to the overall measurement error estimation, the photogrammetric measurement results can be compared with the real 3D coordinates of the points. The measurement error distribution can be determined. With this error distribution, the following equation can then be used to estimate the measurement error for any point on the soil surface. The influence factor on the measurement error can be dependent on the 3D position of the point.

Eₒᵥₑᵣₐₗₗ=f(r,θ,z)

where, Eₒᵥₑᵣₐₗₗ is the error of the photogrammetric measurement method at a specific location on soil surface and r, θ, and z are the coordinates used to define the location of a point in a cylindrical coordinate system.

In use, the material testing device 100 can be used to monitor volume, localized strain, full field temperature, and deformation. The material testing device 100 can be configured to measure volumetric change, local strain development, and temperature variation. The material testing device 100 can further provide data as to local temperature gradients and stress development, which can be used to detect formation of ice lenses in soil samples. Accordingly, the material testing device 100 can facilitate research into mechanisms of frost heave.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A system comprising: a transparent outer cell having an inner surface and an outer surface; a transparent inner cell disposed within the transparent outer cell, the transparent inner cell having an inner surface and an outer surface, wherein the outer surface of the transparent inner cell cooperates with the inner surface of the transparent outer cell to at least partially define a first interior volume, wherein the inner surface of the transparent inner cell at least partially defines a second interior volume, and wherein the second interior volume is configured to receive a material sample; and a plurality of cameras disposed around a circumference of the transparent inner cell, wherein the plurality of cameras are configured to capture visual images and infrared images. wherein each camera of the plurality of cameras is configured to provide captured data to a computing device.

Aspect 2: The system of aspect 1, wherein the plurality of cameras comprises: a first plurality of cameras disposed about the circumference of the transparent inner cell, wherein each camera of the first plurality of cameras is a visual light camera; and a second plurality of cameras disposed about the circumference of the transparent inner cell, wherein each camera of the second plurality of cameras is an infrared camera.

Aspect 3: The system of aspect 1 or aspect 2, further comprising at least one thermocouple disposed within the second interior volume.

Aspect 4: The system of any one of the preceding aspects, wherein the first interior volume is configured to receive pressure controlled air or transparent fluid.

Aspect 5: The system of any one of aspects 2-4, wherein the first and second plurality of cameras are configured to capture spatial data and temperature data, respectively on an entire circumference of the material sample within the second interior volume.

Aspect 6: The system of any one of the preceding aspects, wherein the inner surface of the transparent inner cell has a generally cylindrical profile.

Aspect 7: The system of any one of aspects 2-6, wherein the first plurality of cameras is disposed outside the first interior volume.

Aspect 8: The system of any one of aspects 2-7, wherein the second plurality of cameras is disposed inside the first interior volume and outside the second interior volume.

Aspect 9: The system of any one of the preceding aspects, further comprising a transparent, flexible membrane that is configured to encapsulate at least a portion of the material sample within the second interior volume.

Aspect 10: The system of any one of the preceding aspects, further comprising a top plate disposed at a top of the second interior volume and a bottom plate disposed at a bottom of the second interior volume, wherein the top and bottom plates are configured to apply a temperature gradient across the material sample.

Aspect 11: The system of any one of the preceding aspects, further comprising at least one tensiometer that is configured to attach to the material sample or the inner cell.

Aspect 12: The system of any one of the preceding aspects, further comprising a water access port positioned in fluid communication with the second interior volume.

Aspect 13: The system of aspect 12, further comprising a top plate disposed at a top of the second interior volume and that is configured to expose a top surface of the material sample to a selected temperature.

Aspect 14: The system of any one of aspects 2-13, further comprising at least one processor and a memory in communication with the at least one processor, wherein the memory comprises instructions that, when executed by the at least one processor, cause the at least one processor to: receive image data from the first plurality of cameras; receive thermal image temperature data from the second plurality of cameras; receive thermocouple temperature data from the at least one thermocouple; and log the image data, thermal image temperature data, and thermocouple temperature data in a memory device.

Aspect 15: The system of aspect 14, wherein the memory comprises instructions that, when executed by the at least one processor, cause the at least one processor to: compare the thermocouple temperature data with the thermal image temperature data to create an adjusted thermal image temperature data; and map the image data to the adjusted temperature data.

Aspect 16: The system of aspect 15, wherein the memory comprises instructions that, when executed by the at least one processor, cause the at least one processor to use a photogrammetric method to estimate an error of deformation.

Aspect 17: A method for preparing a material sample for the system of any one of aspects 1-16, comprising printing a pattern on a surface of the material sample.

Aspect 18: A method for preparing a material sample for the system of any one of aspects 1-16, comprising sealing at least a portion of an exterior surface of the material sample in a membrane that is at least one of transparent, flexible, and impermeable.

Aspect 19: The method of aspect 18, wherein the membrane comprises a printed pattern.

Aspect 20: The method of aspect 19, wherein the printed pattern comprises a dot matrix.

Aspect 21: A method comprising: receiving image data from a first plurality of cameras of the system of any one of aspects 2-16, wherein the image data is captured at various intervals of a data capture duration; and processing the data.

Aspect 22: The method of aspect 21, wherein processing the data comprises determining a deformation amount at a given thermal and stress state.

Aspect 23: The method of aspect 21 or aspect 22, wherein processing the data comprises determining a thermal and stress state at which the material sample fractures.

Although several embodiments of the invention have been disclosed in the foregoing specification and the following appendices, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed herein, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A system comprising:
a transparent outer cell having an inner surface and an outer surface;
a transparent inner cell disposed within the transparent outer cell, the transparent inner cell having an inner surface and an outer surface, wherein the outer surface of the transparent inner cell cooperates with the inner surface of the transparent outer cell to at least partially define a first interior volume, wherein the inner surface of the transparent inner cell at least partially defines a second interior volume, and wherein the second interior volume is configured to receive a material sample; and
a plurality of cameras disposed around a circumference of the transparent inner cell, wherein the plurality of cameras are configured to capture visual images and infrared images,
wherein each camera of the plurality of cameras is configured to provide captured data to a computing device.

2. The system of claim 1, wherein the plurality of cameras comprises:
a first plurality of cameras disposed about the circumference of the transparent inner cell, wherein each camera of the first plurality of cameras is a visual light camera; and
a second plurality of cameras disposed about the circumference of the transparent inner cell, wherein each camera of the second plurality of cameras is an infrared camera.

3. The system of claim 2, further comprising at least one thermocouple disposed within the second interior volume.

4. The system of claim 1, wherein the first interior volume is configured to receive pressure controlled air or transparent fluid.

5. The system of claim 2, wherein the first and second plurality of cameras are configured to capture spatial data and temperature data, respectively on an entire circumference of the material sample within the second interior volume.

6. The system of claim 1, wherein the inner surface of the transparent inner cell has a generally cylindrical profile.

7. The system of claim 2, wherein the first plurality of cameras is disposed outside the first interior volume.

8. The system of claim 2, wherein the second plurality of cameras is disposed inside the first interior volume and outside the second interior volume.

9. The system of claim 1, further comprising a transparent, flexible membrane that is configured to encapsulate at least a portion of the material sample within the second interior volume.

10. The system of claim 1, further comprising a top plate disposed at a top of the second interior volume and a bottom plate disposed at a bottom of the second interior volume, wherein the top and bottom plates are configured to apply a temperature gradient across the material sample.

11. The system of claim 1, further comprising at least one tensiometer that is configured to attach to the material sample or the inner cell.

12. The system of claim 1, further comprising a water access port positioned in fluid communication with the second interior volume.

13. The system of claim 12, further comprising a top plate disposed at a top of the second interior volume and that is configured to expose a top surface of the material sample to a selected temperature.

14. The system of claim 3, further comprising: at least one processor and a memory in communication with the at least one processor, wherein the memory comprises instructions that, when executed by the at least one processor, cause the at least one processor to:
receive image data from the first plurality of cameras;
receive thermal image temperature data from the second plurality of cameras;
receive thermocouple temperature data from the at least one thermocouple; and
log the image data, thermal image temperature data, and thermocouple temperature data in a memory device.

15. The system of claim 14, wherein the memory comprises instructions that, when executed by the at least one processor, cause the at least one processor to:
compare the thermocouple temperature data with the thermal image temperature data to create an adjusted thermal image temperature data; and
map the image data to the adjusted temperature data.

16. The system of claim 15, wherein the memory comprises instructions that, when executed by the at least one processor, cause the at least one processor to use a photogrammetric method to estimate an error of deformation.

17. A method comprising:
receiving image data from a plurality of cameras of a system, wherein the image data is captured at various intervals of a data capture duration, wherein the system further comprises:
a transparent outer cell having an inner surface and an outer surface; and
a transparent inner cell disposed within the transparent outer cell, the transparent inner cell having an inner surface and an outer surface, wherein the outer surface of the transparent inner cell cooperates with the inner surface of the transparent outer cell to at least partially define a first interior volume, wherein the inner surface of the transparent inner cell at least partially defines a second interior volume, and wherein the second interior volume is configured to receive a material sample,
wherein the plurality of cameras are disposed around a circumference of the transparent inner cell and configured to capture visual images and infrared images, wherein each camera of the plurality of cameras is configured to provide captured data to a computing device, wherein at least one camera of the plurality of cameras is configured to detect visible light, wherein at least one camera of the plurality of cameras is configured to detect infrared radiation; and
processing the data to determine a deformation amount at a given thermal and stress state.

18. The method of claim 17, further comprising determining a thermal and stress state at which the material sample fractures.

19. The method of claim 17, further comprising: prior to receiving the image data, printing a pattern on a surface of the material sample.

20. The method of claim 17, further comprising: prior to receiving the image data, sealing at least a portion of an exterior surface of the material sample in a membrane that is at least one of transparent, flexible, and impermeable.
